(19) United States
(12) Patent Application Publication
(10) Pub. No.: US 2021/0228224 A1
(43) Pub. Date: Jul. 29, 2021

(54) THROMBECTOMY DEVICES AND METHODS

(71) Applicant: Razack Intellectual Properties, LLC, St. Petersburg, FL (US)

(72) Inventor: Nasser RAZACK, St. Petersburg, FL (US)

(21) Appl. No.: 17/168,166

(22) Filed: Feb. 4, 2021

Related U.S. Application Data
(63) Continuation-in-part of application No. PCT/US2020/062807, filed on Dec. 2, 2020.
(60) Provisional application No. 62/942,652, filed on Dec. 2, 2019.

(51) Int. Cl. A61B 17/221; A61B 17/00; A61B 90/00

(52) U.S. Cl. CPC A61B 17/221; A61B 17/00234; A61B 2090/3966; A61B 2017/00292; A61B 90/39

(57) ABSTRACT

Thrombectomy apparatuses including multiple regions arranged to ensnare and stabilize a thrombus for removal from a blood vessel. Different regions of the apparatus can expand with different radial forces to improve engagement and retention of the thrombus. Different regions of the apparatus can have different diameters for encompassing the clot and for conforming to different portions of a blood vessel. Different regions of the apparatus can have a higher mesh density for capturing or retaining break away material of the clot. A junction region between the different regions of the apparatus can be arranged to enhance expansion and contraction of the apparatus.

### CROSS REFERENCE TO RELATED APPLICATIONS

This patent application claims priority as a continuation-in-part to International patent Application No. PCT/US2020/062807, filed on Dec. 2, 2020 and titled “THROMBECTOMY DEVICES AND METHODS,” which claims priority to U.S. Provisional Patent Application No. 62/942,652, titled “THROMBECTOMY DEVICES AND METHODS” and filed on Dec. 2, 2019, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Thrombectomy devices and methods of their use are described. In particular, apparatuses for ensnaring and removing thrombi are described.

### BACKGROUND

Approximately fifteen million people suffer stroke worldwide each year, with five million people dying and another five million permanently disabled. Ischemic strokes represent the fifth most common cause of death in western world and the number one cause of long-term disability. Acute treatments for ischemic stroke include thrombolysis, which involves administering medication, typically intravenously, to breakdown blood clots (thrombi) in the patient's blood vessels. When administered within three hours of symptom onset, systemic intravenous thrombolysis have found to result in an overall benefit of 10% with respect to living without disability. Results from administering systemic intravenous thrombolysis after three hours are less clear, and longer periods before administration have shown to even worsen outcomes.

More recent interventions include mechanical thrombectomy procedures, in which the blood clot causing the ischemic stroke is removed from the blood vessel, typically performed on a large artery such as the middle cerebral artery. Stent-retriever catheters devices, in particular, have demonstrated to be safe and effective, improve outcomes and reduce mortality for patients. Stent-retriever devices generally employ a stent and catheter technology to entrap the blood clot and to retrieve the blood clot from the blood vessel. Other mechanical thrombectomy technologies include direct aspiration, where an aspiration catheter is advanced through the occluded vessel and direct aspiration is applied to retrieve the thrombus. In some cases, direct aspiration is combined with a stent-retriever, which may have additional benefits.

Despite the tremendous technological advancement for large vessel occlusion acute stroke intervention, there remain numerous challenges for existing neurointerventional devices. Currently, about 20% to 30% of thrombi are resistant to current mechanical thrombectomy approaches. Factors limiting the success of retrieval technology include sufficiently engaging the device within the thrombus to penetrate the thrombus and hold it, sufficient incubation time and maximizing the greatest effect of approval force when removing the device. Because mechanical thrombectomy devices are typically larger than aspiration catheters, when utilizing this technology together, the thrombus must deform to a smaller size to fit within the aspiration catheter. Alternatively, the thrombus can be sheared outside of the aspiration catheter causing potential fragmentation resulting in downstream emboli. Thus, developing successful retriever technology that can sufficiently engage and hold onto the thrombus that can also reconfigure and simultaneously hold the thrombus as it is pulled into the aspiration catheter would improve first pass revascularization and complete reperfusion rates.

### SUMMARY

Described herein are thrombectomy apparatuses (e.g., devices, systems, etc.) and methods, which may be used as part of a neuroendovascular procedure to remove a thrombus. The apparatuses can include a clot capture portion that expands within a patient's blood vessel to penetrate through and engage with the thrombus. Once sufficiently engaged with the thrombus, the apparatus can be retrieved from the vessel along with the captured or entrain thrombus, thereby removing the thrombus from the patient's body. The clot capture region may be used in combination with one or more coaxial catheters to retain the apparatus (e.g., the clot capture region) in a contracted state while the clot capture region is passed inside the arterial circulation, and to release the clot capture region in an expanded state at or near the thrombus. In some cases, a microcatheter is used to house the clot capture region and keep the clot capture region in a contracted state, and a guide catheter is used to guide the microcatheter (with the clot capture region housed therein) to the target site in the blood vessel. In some instances, the microcatheter and/or a distal wire (e.g., a distal microwire) is used to penetrate through the thrombus and to position at least a portion of the clot capture region distally past the thrombus (e.g., prior to expansion of the clot capture region). The distal wire may also be configured to assist in tracking within the catheter and/or vessel. In some cases, an aspiration catheter is used in combination with the clot capture region.

The clot capture region can include different regions that are arranged to apply differing amounts of expansion force against the thrombus and/or blood vessel walls. The clot capture region can include a clot engagement region having a higher expansion force that provides sufficiently high expansion force to penetrate through the thrombus and press the clot engagement region against the blood vessel walls, which may lift some of the thrombus material from the blood vessel wall. A lower expansion force region of the clot capture region, e.g., a distal capture region, positioned distal to the higher expansion force (clot engagement) region, can retain some of the dislodged thrombus material during the expansion of the clot capture region, thereby stabilizing the thrombus.

Different regions of the clot capture region can have different shapes, sizes and geometries according to their respective functions for engaging with and stabilizing the thrombus. A first portion of the clot capture region, e.g., the clot engagement region, can have a relatively large diameter when expanded to facilitate entrapment of the thrombus. For example, the larger diameter can help to fully encompass the thrombus. In some cases, the first portion has an outer diameter that is larger than the inner diameter of the blood vessel. The clot capture region may have a second portion, e.g., a distal capture region, having a smaller outer diameter than the first portion. The smaller diameter may allow the second portion to fit within smaller portions of the blood vessel, such as branching portions of the blood vessel. In some cases, one or both of the first and second portions of the clot capture region have a tapered profile or one or more curved outer surfaces. In some cases, the first portion of the clot capture region has a larger opening area (e.g., less dense wire structure) compared to the second portion of the clot capture region. This may allow the first portion to accommodate the thrombus material within the lumen of the clot capture region. The smaller opening area (e.g., more dense wire structure) of the second portion of the clot capture region may allow the second portion to retain any dislodged thrombus material from the thrombus.

The devices described herein can include one or more markers that may be visible to a practitioner during placement, expansion and/or retrieval of the device from the patient. The markers may be visible using any radiology techniques. In some examples, the markers are radiopaque markers that are visible using fluoroscopy (e.g., x-ray fluoroscopy) techniques. The radiopaque markers may be made of any radiopaque material suitable for endovascular procedures (e.g., biocompatible). The markers may be placed along strategic locations of the device so that the practitioner can visualize the full length and/or wide (diameter) of the device. For example, the markers can be placed at the proximal end and/or the distal end of the clot capture region. In some cases, the markers are placed the largest diameter of the clot capture region. The markers may have any shape (e.g., ring-shaped, spherical, wire, or hook).

In some examples, a catheter thrombectomy device includes a clot capture region, where the clot capture region includes: a proximal first region configured to expand with a first radial force; and a distal second region coupled to the first region, the first region configured to expand with a second radial force that is less than the first radial force. The second region may taper distally toward a distal end of the second region. The first region and the second region may be connected at a junction region of the clot capture region, wherein struts of the first region are fused together (or continuous) with struts of the second region at the junction region. The first region and the second region may be connected at a junction region of the clot capture region, wherein struts of the first region are linked (or continuous) with struts of the second region at the junction region. The first region and the second region may be connected at a junction region of the clot capture region, wherein the first region and the second region overlap at the junction region. The first region may have a longer length than the second region. A distal end of the second region may have a curved shape. A distal end of the second region may include a dimple.

In some examples, a catheter thrombectomy device includes a clot capture region, where the clot capture region includes a proximal first region having a first strut spacing; and a distal second region coupled to the first region, the second region having a second strut spacing that is less than the first strut spacing.

In some examples, a catheter thrombectomy device includes a clot capture region, where the clot capture region includes a distal second region; and a proximal first region coupled to the second region at a junction region of the clot capture region, the first region having an outer diameter that tapers from a first diameter at a proximal end of the first region to a second diameter at the junction region, the second diameter greater than the first diameter. The second region may taper distally toward a distal end of the second region. A distal end of the second region may have a curved shape. A distal end of the second region may include a dimple.

For example a thrombectomy apparatus may include: a clot engagement region comprising a first region of a plurality of struts and configured to expand with a first radial force; a tapered distal capture region comprising a porous polymeric membrane integrally formed onto a second region of a plurality of struts at a distal end of the clot engagement region, so that the struts are embedded within the polymeric membrane, the distal capture region configured to expand radially with a second radial force; and a distal microwire extending from the tapered distal end region by between 0.5 cm and 10 cm. The second radial force may be less than the first radial force.

Any of the apparatuses described herein may include a clot engagement region that is tapered to have a greater radial diameter at the distal end of the clot engagement region. The porous polymeric membrane may have a porosity that is greater than 50 microns (e.g., between 50 microns and 1 mm, between 100 microns and 1 mm, greater than 150 microns, greater than 200 microns, greater than 300 microns, greater than 400 microns, greater than 500 microns, etc.).

The clot engagement region and the distal capture region may be continuous. In some examples, the clot engagement region and the distal capture region are connected at a junction region, wherein struts of the first region are fused with struts of the second region at the junction region. The clot engagement region may be configured as a stent. The clot engagement region and the distal capture region may be connected at a junction region of the stent, wherein the clot engagement region and the distal capture region may overlap at the junction region.

In any of these examples, the clot engagement region may have a longer length than the distal capture region. The distal end of the distal capture region may have a curved shape. The distal end of the distal capture region may include a dimple.

Any of these apparatuses may include a first plurality of radio opaque markers at a proximal end of the clot engagement region and a second plurality of radio opaque markers at a junction between the clot engagement region and the distal capture region. Any of these apparatuses may include an elongated support extending through the clot engagement region and the distal capture region and coupled to or continuous with a distal end of the distal capture region (e.g., the distal wire).

In some examples the clot engagement region is between 5 and 40 mm long and is between about 1.1 and 2 times the length of the distal capture region. The maximum radial diameter of the clot engagement region may be between 4 mm and 8 mm in an unconstrained state. The apparatus may include a deliver catheter or sheath. The apparatus may include a lubricious coating at the distal end region.

Also described herein are methods of removing a clot from a blood vessel. For example, a method of removing a clot from a blood vessel may include: passing a thrombectomy apparatus in a collapsed and constrained state through the clot within the blood vessel, wherein the thrombectomy apparatus comprises: a clot engagement region comprising a plurality of struts wherein the clot, a tapered distal capture region comprising a porous region at a distal end of the clot engagement region, wherein the plurality of struts are continuous with the porous region, and wherein pores of the porous region are smaller than gaps between the struts of the clot engagement region, and a distal microwire extending from the tapered distal capture region by between 0.5 cm and 10 cm. The device may be pushed through the clot with the distal microwire (which may be sharp, e.g., pointed). The device may be driven by a proximal pusher. The device may be held in a constrained (collapsed) configuration within a catheter or tube that may also be driven at least partially (or in some cases, completely) through the clot.

Once the device is extended into and/or past the clot it may be expanded within the blood vessel and/or within the clot. For example, the method may include expanding the clot engagement region within the blood vessel with a first radial expansion force and expanding the tapered distal capture region within the blood vessel with a second radial expansion force, so that the clot engagement region expands through the clot, wherein the clot engagement region expands with a greater radial expansion force than the tapered distal capture region. In some examples the device may be positioned in the blood vessel so that the distal capture region is extended past the clot, while the proximal clot engagement region is expanded within the clot. The process may be guided by fluoroscopy using one or more makers on the device (e.g., indicating the distal end of the device, or the entire device may be configured for imaging under fluoroscopy.

Once expanded, the device may be pulled proximally to draw the clot (or a portion of the clot) into the capture region the device with captured clot may be pulled back through the body. In some examples the device is at least partially collapsed back into a catheter (either the original delivery catheter or a second recapture catheter that has a larger diameter than the first catheter.

Such methods may include inserting a clot capture region while in a contracted (e.g., collapsed and constrained) state through the clot within the blood vessel, the clot capture region including a proximal first region coupled to a distal second region; and causing the clot capture region to expand within the blood vessel so that at least a portion of the first region expands into the clot, wherein the first region expands with a greater radial force than the second region. The method may further include pulling the clot capture region proximally to remove the clot capture region with the clot from the blood vessel. The second region may retain material from the clot during expansion of the clot capture region, during removal of the clot capture region from the blood vessel, or during expansion and removal of the clot capture region from the blood vessel. The first region may have a larger strut spacing than the second region. The first region may be coupled to the distal second region at a junction region of the clot capture region, wherein the first region has an outer diameter that tapers from a first diameter at a proximal end of the first region to a second diameter at the junction region, the second diameter greater than the first diameter. Causing the clot capture region to expand within the blood vessel may include pulling a catheter sheath housing the clot capture region proximally to cause the clot capture region to expand, wherein the greater expansion force of the first region causes the first region to be pinned against walls of the blood vessel, thereby holding the clot capture region in place within the blood vessel.

These and other examples and aspects are described herein.

### BRIEF DESCRIPTION OF THE DRAWINGS

Novel features of examples described herein are set forth with particularity in the appended claims. A better understanding of the features and advantages of the examples may be obtained by reference to the following detailed description that sets forth illustrative examples and the accompanying drawings.

FIG. 1 shows a side view of an example thrombectomy device having a stent with multiple regions.

FIGS. 2A and 2B show side views of another example of a thrombectomy device.

FIGS. 3A-3E show a thrombectomy device being used to remove a clot from a blood vessel.

FIG. 4 illustrates another example of a thrombectomy device having a stent with multiple regions as described herein. In this example, the stent includes a distal region that is tethered to the proximal region.

FIG. 5 is a schematic of another example of a thrombectomy device as described herein.

FIG. 6 illustrates another schematic example of a thrombectomy device, showing exemplary dimensions.

FIG. 7 shows one example of a clot capture region formed as a two-dimensional sheet which may then be rolled to form the body of the clot capture region, including the proximal end for attaching to a puller (e.g., pull wire, rod, etc.), and the distal end that may be formed into a tapered region and coated with a polymeric material to form the distal capture region as described herein.

FIGS. 8A-8C illustrate one example of a thrombectomy device as described herein. FIG. 8A shows the entire device. FIG. 8B shows an enlarged view of the distal end of the device. FIG. 8C shows an enlarged view of the proximal end region of the device.

FIG. 9 illustrates one example of a thrombectomy device as described herein.

### DETAILED DESCRIPTION

Described herein are apparatuses (e.g., thrombectomy apparatuses) that are configured to provide a high radial force in order to penetrate into and secure a clot, so that the clot may be stabilized and removed from the vessel. In some examples these apparatuses may be configured as catheter devices. These apparatuses may include different features to improve removal of a thrombus from a patient's blood vessel. The apparatuses can include an expandable stent portion with various regions of having different aspects, such as different diameters, mesh densities, expansion forces and/or outer shapes.

In general, these apparatuses may include a distal capture region configured as a lower porosity region (e.g., a mesh, such as a Nitinol mesh) that is configured to taper distally. In some examples this taper may be rounded towards the distal end. The distal capture region may have a first porosity (in some examples, a mesh density, a number of mesh strands or filaments, etc.). One or more radiopaque marker may be coupled to the distal end of the distal capture region. In some examples the distal end of the distal capture region may be configured to engage with a support or push wire (e.g., guidewire, etc.) to hold the distal end of the apparatus in position within a vessel when deploying the apparatus, as will be described below. The proximal end may have a higher porosity (e.g., more openings, larger openings, etc.) as compared to the distal capture region (distal end).

The distal capture region may be, e.g., between 5 and 60 mm long (e.g., between about 5 and 30 mm, long, between about 10 and 25 mm long, between about 15 and 22 mm long, etc.). As described below, the length of the distal capture region may be less than or equal to (e.g. preferably less than) the length of the adjacent clot engaging region. In some examples, the distal capture region is a membrane, such as a polyurethane membrane.

The apparatus may also include a clot engaging region adjacent to (either directly or indirectly) and coupled with the proximal end of the distal capture region. The engagement region may be configured to expand radially outward with an expansion force that is greater than the expansion force of the distal capture region. The engagement region may be configured to taper proximally, so that the distal-most end of the engagement region is larger (by between about 1.05× and 2×, between about 1.1× and about 1.5×, between about 1.1× and 1.4×, between about 1.1× and 1.3×, between about 1.1× and about 1.2×, between about 1.1× and 1.15×, etc.) the diameter of the proximal end of the clot engagement region. The clot engagement region may increase in radial diameter along the proximal-to-distal axis in a linear manner, or in an increasing manner (e.g., exponentially increasing). Thus the outer profile of the clot engagement region may have a funnel-shape, a bell-shaped, or the like.

The maximum outer diameter of the clot engagement region (in an unconstrained configuration) may be, e.g., between about 3 and about 10 mm (e.g., between about 4 and about 8 mm, between about 5 and about 7 mm, 4 mm or greater, 5 mm or greater, 6 mm or greater, 7 mm or greater, 8 mm or greater, 9 mm or greater, 10 mm or greater, etc.). In some examples the maximum outer diameter of the clot engagement region may be at the distal most end of the clot engagement region.

The clot engagement region may be formed of one or more fibers, e.g., Nitinol or other fibers, and is/are configured to radially self-expand upon release from the delivery (e.g., micro-) catheter. In some examples the engagement region may be a stent or a stent-like element. The engagement region may be configured to expand radially with a force of between about 0.1 N to about 5 N (e.g., between about 0.1 N to about 4 N, between about 0.1 N to about 3 N, between about 0.1 N to about 2.5 N, between about 0.1 N to about 2 N, between about 0.1 N to about 1.5 N, 0.1 N or greater, 0.2 N or greater, 0.5 N or greater, 0.75 N or greater, 1 N or greater, 1.1 N or greater, etc.). The radial force may be distributed along the length of the engagement region uniformly, or it may be greater distally (e.g., the radial force may increase from proximal to distal along the engagement region. Alternatively, in some examples the radial force may increase from distal to proximal, so that the radial force is greater proximally.

The clot engagement region may be formed of one or more material. In some examples the clot engagement region is configured as a stent or stent-like structure, which may be formed from a sheet of material (e.g., Nitinol, stainless steel, etc.). In some examples the clot engagement region is formed of a knitted, woven, or braided material (e.g., filaments of material, such as knitted, braided and/or woven Nitinol, stainless steel, etc.). In general, the clot engagement region may have a mesh density (and/or a porosity, number of mesh strands or filaments, etc.) that is greater than that of the distal capture region. For example, the clot engagement region may have a porosity that is greater than the porosity of the distal capture region. For example, the clot engagement region may have a porosity that is greater than 1.5× the porosity of the distal capture region (e.g., between about 1.5× and 30×, between about 1.5× and 20×, between 2× and 25×, between 2× and 20×, between 2× and 10×, 2× or greater, 3× or greater, 5× or greater, 7.5× or greater, 10× or greater, 15× or greater, 20× or greater, etc.). The relative porosity differences between the clot engagement region and the distal capture region may enhance the clot capture, e.g., helping entangle the clot in the more proximal clot engagement region.

The cot engagement region may be any appropriate length, including, e.g., between about 5 mm and about 60 mm (e.g., between about 5 mm and 50 mm, between about 5 mm and 45 mm, between about 5 mm and 40 mm, between about 5 mm and 35 mm, between about 5 mm and 30 mm, 5 mm or greater, 12 mm or greater, 15 mm or greater, 20 mm or greater, 25 mm or greater, 30 mm or greater, 35 mm or greater, 40 mm or greater, 45 mm or greater, 50 mm or greater, 55 mm or greater, 60 mm or greater, etc.). The clot engagement region may be longer than the distal capture region. For example, the clot engagement region may be between 1.1× and 5× the length of the distal capture region (e.g., between 1.1× and 4×, between about 1.1× and 3×, between about 1.1× and 2×, between about 1.1× and 1.5×, about 1.1× or greater, about 1.2× or greater, about 1.3× or greater, about 1.5× or greater, about 1.6× or greater, about 1.7× or greater, about 1.8× or greater, about 1.9× or greater, about 2× or greater, about 2.5× or greater, about 3× or greater, etc.). The relative length differences between the clot engagement region and the distal capture region may enhance the clot capture, e.g., allowing capture of the clot while preventing the clot from escaping from the clot capture region when withdrawing the apparatus with capture clot material.

The interface between the clot engagement region and the distal capture region may include one or more (and preferably 2 or more, 3 or more, 4 or more, etc.) radiopaque markers. This interface or transition region between the clot engagement region and the distal capture region is the region at which the clot engagement region and the distal capture region may have the same radial outer diameter. In some examples the radiopaque marker(s) are arranged as a continuous ring around the radius of the apparatus. In some examples a plurality of radiopaque markers are arranged at radial positions around the radius of the interface region at the same longitudinal position of the apparatus. In some examples the outer surface of the interface region and/or the clot engagement region may include a lubricious material (e.g., expanded PTFE, and/or lubricant, etc.). The outer surface of the clot engagement region may be configured to allow sliding within a delivery catheter.

The proximal end of the clot engagement region may also include one or more (e.g., 2 or more, 3 or more, 4 or more, etc.) radiopaque markers. This proximal end of the clot engagement region may couple to one or more pull elements (e.g., strands, wires, rods, fibers, etc.) that may extend proximally, e.g., within the delivery catheter (e.g., microcatheter). In some examples the radiopaque marker(s) are arranged as a continuous ring around the radius of the apparatus at this longitudinal position. In some examples a plurality of radiopaque markers are arranged at radial positions around the radius of the proximal end of the clot capture region at the same longitudinal position.

In any of these examples a central support (e.g., a push wire, filament, strand, rod, etc.) may extend through the central region of the apparatus, including through the clot engagement region and distal capture region. This central support may be configured as mentioned above to hold the distal end of the apparatus in a selected position within the vessel when withdrawing the delivery catheter (e.g., microcatheter) and/or when pushing the apparatus distally to deploy the apparatus within the vessel. In some examples the central support is part of or coupled to the one or more pull elements coupled to the proximal end of the clot engagement region. The central support may have a sufficient column strength so as to allow the deployment of the apparatus from the delivery catheter (e.g. microcatheter).

As mentioned above, any of these apparatuses, including systems or devices that also include the thrombectomy devices with clot engagement regions and distal capture regions as described above, may also include a deliver catheter, which may be a microcatheter (e.g., for use in the neurovasculature). The deliver catheter may be configured to constrain the thrombectomy apparatus (e.g., thrombectomy device) described above in a delivery configuration while allowing it to be distally deployed from out of the delivery catheter so that at least the clot engagement region has a high radial force as described above. In some examples the inner surface of the delivery catheter may include a lubricious material. For example, the delivery catheter may be formed of a material that is highly lubricious such as expanded PTFE (e.g., “Teflon”); in some examples the delivery catheter is coated internally with a lubricous material.

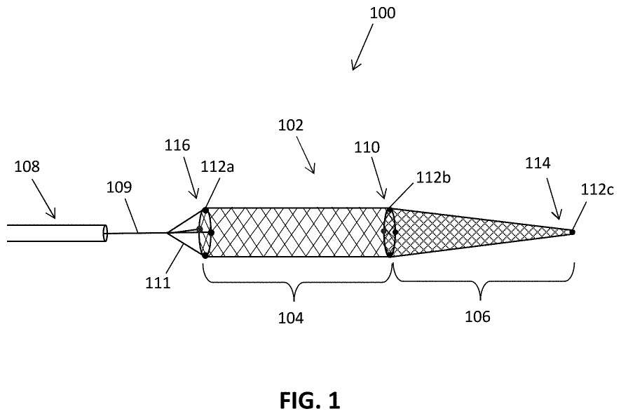

FIG. 1 shows one example of a thrombectomy device 100, which includes a clot capture region 102 at the distal end, having a generally tubular shape. In some examples, at least a portion of the clot capture region 102 is biased to maintain an expanded state and to assume a contracted state upon an applied inward radial force. For example, a delivery catheter or sheath 108 can be placed around the outer diameter of the clot capture region 102 and apply sufficient inward radial pressure to contract the clot capture region 102 in a contracted, smaller diameter delivery state. The delivery catheter or sheath 108 can be used to position the clot capture region 102 within the lumen of a blood vessel. The clot capture region 102 can be biased to radially expand to an expanded (e.g., deployed) state once released from the deliver catheter or sheath 108, as shown in FIG. 1 (showing the apparatus in a relaxed, fully deployed state). Within a vessel, the apparatus, and particularly the clot engagement region 104 may be constrained from fully expanding by the vessel wall.

The clot capture region 102 can include a proximal clot engagement region 104 and a distal capture region 106; either or both of these regions may be formed as a scaffold of struts (e.g., wires) separated by spaces, which allow the clot capture region 102 to radially expand and contract. In some examples the clot engagement region 104 may be configured as a stent. The network of struts forming either or both the clot engagement region and/or the distal capture region 106 can be arranged in any pattern. For example, the clot capture region 102 can include a clot engagement region 104 formed as a mesh stent, annular coil stent, tubular stent, ganglion stent, or any combination thereof. Once the distal end of the clot capture region and/or delivery catheter/sheath 108 is positioned in a desired location within the blood vessel, typically through a clot, the clot capture region 102 can be deployed from the catheter/sheath 108, e.g., by pulling the catheter/sheath proximally and/or driving the clot capture region distally to extend the clot capture region from the catheter/sheath 108 allowing the clot capture region to expand radially into and through the clot at the clot engagement region 104.

The clot engagement region 104 (which may also be referred to as an entrapment region) is typically adjacent to a distal capture region 106 (also referred to as a capture region). When the clot capture region 102 is placed in the blood vessel, the clot engagement region 104 can be positioned proximally relative to the distal capture region 106 with respect to the catheter/sheath 108 so that the clot engagement region 104 can engage with and ensnare an obstruction (e.g., thrombus/clot). The clot capture region 102 can be connected proximally to a pull wire 109 (e.g., guidewire) for retracting the clot capture region 102; as mentioned this pull wire may also be configured as a support (or may be coupled to a support) such as a wire, that may help hold the distal end of the device (e.g., the distal capture region 106) in place during deployment. The wire 109 can also be used to pull the clot capture region 102 proximally with the entrapped obstruction, thereby removing the obstruction from the blood vessel, and into the delivery catheter/sheath and/or a separate capture sheath or catheter than may be passed over the deployment catheter or sheath 108. The capture region 106 can be configured to capture and retain the obstruction within the clot capture region 102, or otherwise engaged with the clot, as the clot capture region 102 is pulled proximally and retrieved from the vessel. In FIG. 1, the distal capture region 106 is shown as a mesh having a tighter mesh pattern (e.g., smaller pores) than the clot engagement region; in some examples the distal capture region 106 is a membrane, including a porous membrane.

The clot engagement region 104 and the distal capture region 106 may include different aspects in order to improve engagement and/or retention of the obstruction. For example, in some examples, the clot engagement region 104 is configured to expand with a greater radial force than the distal capture region 106. The greater expansion force can allow the struts of the clot engagement region 104 to sufficiently push through the obstruction and to entrap at least a portion of the obstruction within the lumen of the clot engagement region 104. For example, the relatively high expansion force of the clot engagement region 104 can compensate for the flexibility and compliance of the blood vessel walls. In some cases, the clot engagement region 104 is oversized (has larger outer diameter when expanded) compared to the inner diameter of the blood vessel to facilitate the entrapment. This can allow the clot engagement region 104 to conform to the vessel walls around it and more fully encompass the obstruction. The magnitude of the radial forces may vary depending, for example, on the size and shape of the clot engagement region 104 and/or on the size of the vessel. The distal capture region 106 may have a lower expansion force and/or smaller average diameter.

The clot engagement region 104 and the distal capture region 106 may have different strut densities. That is, the area between the struts (e.g., mesh wires) may vary; this may also be referred to as the porosity of the clot engagement region and the porosity of the distal capture region. The area between the struts may be referred to as gaps or pores. In some cases, the area of space between the struts in the clot engagement region 104 is greater than that of the distal capture region 106. For example, the distal capture region 106 can have a greater mesh density (and therefore porosity) than the entrapment region. This can allow more space for the occlusion to enter the clot engagement region 104. Once the clot engagement region 104 is engaged against an occlusion, the smaller spaces between the struts of the distal capture region 106 can prevent (or reduce the occurrence of) escape of occlusion material as the stent 112 is pulled proximally. In some cases, the distal capture region 106 prevents (or reduce the occurrence of) the occlusion material from breaking away from the clot capture region 102.

In some cases, the distal capture region 106 narrows to a smaller diameter at a distal end 114. This tapered shape may allow the capture region 106 to fit within more confined spaces of a blood vessel. For example, the smaller distal end 114 may allow the distal capture region 106 to temporarily reside within smaller branches of at more distal portions of a blood vessel system. In some instances, the distal end 114 of the distal capture region 106 may be closed (e.g., pinched off).

The size (e.g., diameter and length) of the clot capture region 102 can vary depending, in part, on the inner diameter of the target blood vessel (e.g., the vessel into which the apparatus is to be inserted). In one example the outer diameter of the clot engagement region 104 is about 6 millimeters (mm) when expanded, and small enough to fit within a 0.27 mm diameter delivery catheter/sheath 108. In some examples, the clot engagement region 104 is longer than the distal capture region 106. For example, in one examples the length of the clot engagement region 104 is about 30 mm and the length of the distal capture region 106 is about 20 mm.

The material of the clot capture region 102 may vary. In some cases, the clot capture region 102 is made of a metal (e.g., platinum, Nitinol, stainless steel and/or titanium). In some cases, the clot capture region 102 is made of a polymer material. In some cases, the clot capture region 102 is made of a combination of different materials (e.g., metal and polymer). In some cases, the clot engagement region 104 and the distal capture region 106 are made of the same material(s). In some examples, the clot engagement region 104 and the distal capture region 106 are made of different materials. In some examples, the struts of the distal capture region 106 are nitinol mesh and the struts of the clot engagement region 104 are made of platinum and/or nitinol.

The clot engagement region 104 and the distal capture region 106 may be connected at a junction region (e.g., intermediate or transition region) 110. At the junction region 110, the struts of the clot engagement region 104 may be joined to the struts of the distal capture region 106 using any suitable techniques. For example, the struts may be fused together (e.g., welded). In some cases, the struts (wires) of the clot engagement region 104 and distal capture region 106 movably engage with each other. For example, the wires struts of the clot engagement region 104 can be linked or woven to wires struts of the distal capture region 106. The struts can be connected such that the clot capture region 102 can properly expand and collapse. The junction region may be arranged to enhance expansion and/or contraction of the clot capture region 102. In some instances, the clot engagement region 104 overlaps with the distal capture region 106 at the junction region 110. In other cases, the struts of the clot engagement region 104 and the distal capture region 106 are fused together at the junction region 110 without overlapping.

In some examples, the clot capture region 102 includes one or more radiopaque markers (e.g., 112a, 112b and 112c) used to provide visibility of the clot capture region 102 under, for example, x-ray fluoroscope during the thrombectomy procedure. The radiopaque marker(s) may be place in select locations of the clot capture region 102 so that diameter and length of the clot capture region 102 can be visualized as it is being manipulated within a blood vessel. The radiopaque marker(s) may be small distinct objects of any shape. In some cases the radiopaque marker(s) are small radiopaque objects placed around the diameter of the clot capture region 102 at various locations along the clot capture region 102, such as the proximal end 116, the distal end 114 and the junction region 110. The shape of the radiopaque markers can vary. For example, the radiopaque markers may have an approximately spherical shape, polyhedron (e.g., cube, hexahedron), or ring shape. If ring shaped, the radiopaque markers may be placed around various portions along the length of the stent 102.

The puller (e.g., wire) 109 can be connected to any portion of the clot capture region 102. In some examples, the puller 109 is connected to a proximal end 116 of the clot engagement region 104 by one or more tethers 111, as shown in FIG. 1. In other cases, the puller 109 runs at least partially through the inner lumen of the clot capture region 102 and connects to one or more portions of the clot capture region 102, such as the junction region 110 and/or the distal end 114.

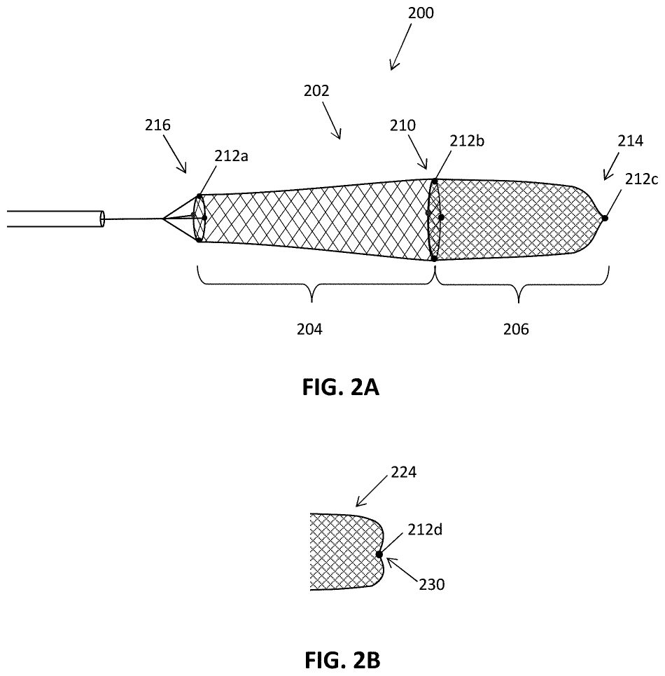

FIGS. 2A and 2B show a thrombectomy apparatus 200 including a clot capture region 202. The clot capture region 202 includes a clot engagement region 204 and a distal capture region 206 similar to the clot engagement region 104 and distal capture region 106, respectively of the example shown in FIG. 1, except that the clot engagement region 204 and a distal capture region 206 can have different shapes. The proximal end 216 of the clot engagement region 204 can have a first diameter that tapers to a second diameter larger than the first diameter at the distal end of the clot engagement region 204 (at the junction region 210). The larger second diameter may cause the junction region 210 to apply greater radial force along the blood vessel wall, which can help to lift the obstruction off the blood vessel wall. For example, the proximal end 216 of the clot engagement region 204 may have a radial diameter of about 5 mm and the distal end of the clot engagement region 204 (at the junction region 210) may have a diameter of about 7 mm. The distal capture region 206 can have a wide (e.g., not tapered) distal end 214, which may help retain the obstruction material therein. The distal capture region's distal end 214 may also be curved so that it may glance off the blood vessel walls for better maneuverability and less injury to the vessel walls. FIG. 2B an example of one example of a distal capture region having a curved distal end 224 that forms a dimple (indentation). The clot capture region 202 can include one or more radiopaque markers 212a, 212b, 212c and/or 212d at different regions of the clot capture region 202. For example, in some examples, the apparatus includes four radiopaque markers (e.g., beads) around the perimeter at various locations, such as the proximal region and/or medial junction region 210.

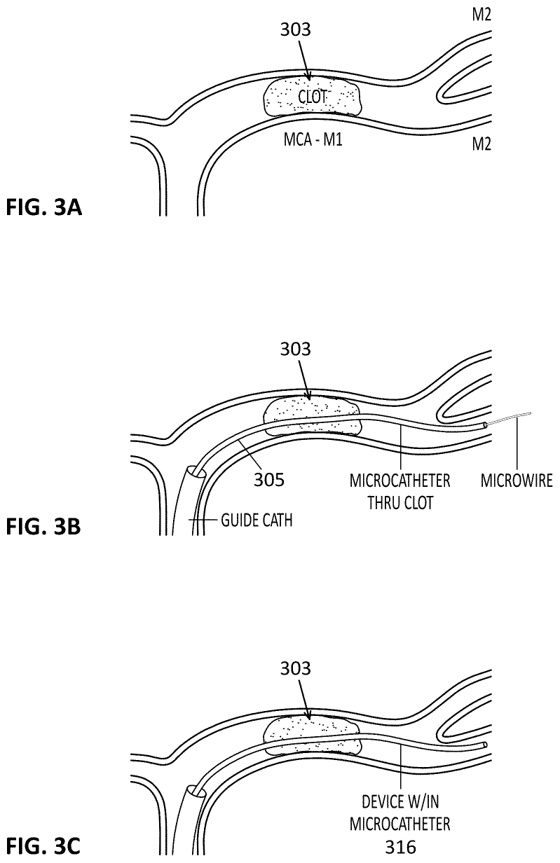

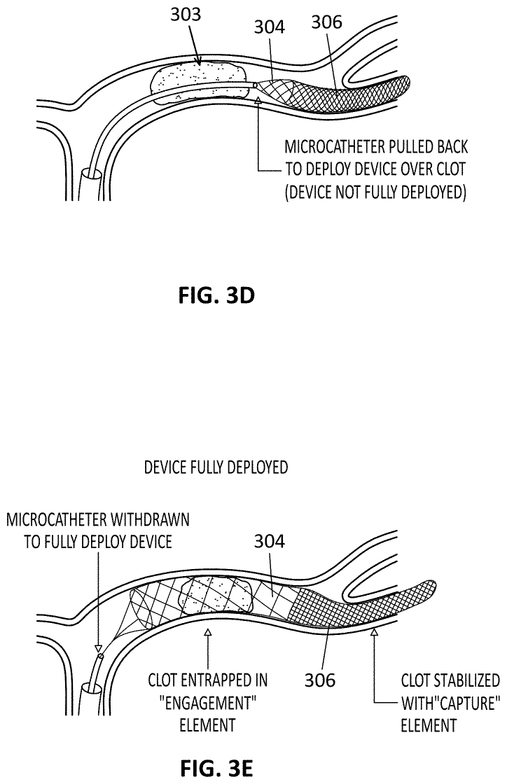

FIGS. 3A-3E illustrate an example method of using a thrombectomy apparatus according to some examples. FIG. 3A shows a clot 303 causing blood flow obstruction within a blood vessel, in this case, the middle cerebral artery (MCA) M1 that branches off at two smaller arteries M2. In FIG. 3B, the clot 303 is penetrated by a wire (microwire) 305 and/or microcatheter (or just the microcatheter). In some cases, a guide catheter is used to guide the microcatheter to the clot. The microcatheter can include a clot capture region of a device 316 in a contracted state therein, as shown in FIG. 3C. In FIG. 3D the microcatheter is pulled back to allow the second region (the distal capture region 306) to expand and the first region (the clot engagement region 304) starting to expand into and through the clot. As shown, the smaller diameter and/or tapered second region can allow the second region to expand within a smaller artery M2. The greater expansion force of the first (clot engagement) region can cause the first region to be pinned against the blood vessel walls, thereby holding the device in place as the microcatheter continues to be pulled back. FIG. 3E shows the first 306 and second 304 (clot engagement) regions fully expanded, with the first region expanded into the clot. The first region can penetrate through and entrap the clot, and the second region can stabilize the clot as the device expands into the clot. After the stent is sufficiently engaged with the clot, the device can be pulled proximally by a pull wire to remove the device and the clot from the blood vessel. In some cases, the apparatus is configured to at least partially contract to a smaller diameter as the wire(s) are pulled. In some cases, the apparatus is pulled and at least partially withdrawn into the guide catheter. In some examples, removal of the clot is assisted using aspiration, such as from an aspiration catheter.

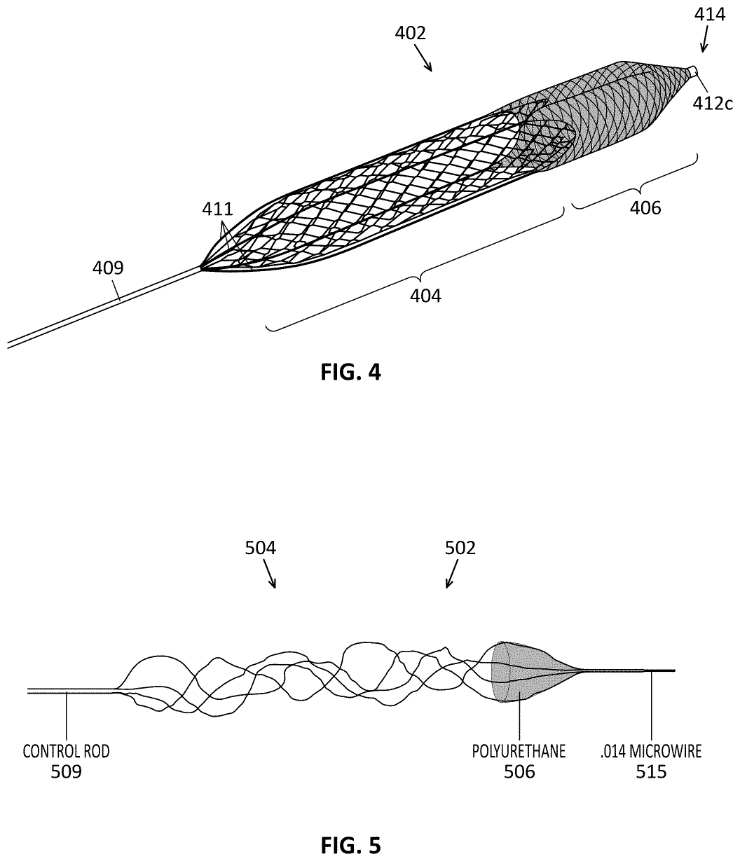

FIG. 4 shows one example of a thrombectomy device formed as a distal braided basket with a laser cut capture stent; the braided basket forming the distal capture region 406 has tethers 411 that reach back to the proximal control rod 409 (e.g., pull wire) for securement. The clot engagement region 404. The device includes a clot capture region 402 that may encompass both the proximal clot engagement region 404 and a distal capture region 406. As describe above, the clot capture region may be configured to expand. The distal capture region 406 narrows, shown in this example as a taper, to a smaller diameter at a distal end 414. This tapered shape may allow the distal capture region 406 to fit within more confined spaces of a blood vessel. For example, the smaller distal end 414 may allow the distal capture region 406 to temporarily reside within smaller branches of at more distal portions of a blood vessel system. In some instances, the distal end 414 of the distal capture region 406 may be closed (e.g., pinched off). In FIG. 4, the distal end also includes a marker 412c (e.g., a radiopaque marker).

Any of the apparatuses described herein may include a capture region that is formed of a polymeric material, as described above. For example, the apparatus may include a polymeric (e.g., polyurethane) coating forming the distal capture region. The distal capture region may be fixed relative to the capture portion and may be formed onto the distal end of the clot engagement region. For example, the distal capture region may include a polymeric membrane that is formed integrally onto over overtop of (and/or encapsulating) the material (e.g., metal, such as Nitinol) forming the clot capture region. Thus when the clot capture region includes a stent-like structure (as shown in FIGS. 4-5), the stent may be tapered (and in some examples, gathered) at the distal end region to form a conical tip region at the distal end. The struts may therefore form windows, and a coating may be applied to these windows and over the distal end of the struts forming the distal capture region. For example, a mandrel may be used to provide a temporary support for the coating to form onto, which may then be removed. Pores may then be formed into this distal capture region. For example, pores may be formed by laser drilling or cutting. Such examples may be particularly advantageous because they may be all one piece (e.g., unitary), which may provide a stronger structure without requiring joints or welds. This example may also be readily manufactured, as it does not require additional components, or actuations necessary for the distal filter to be placed or opened.

FIG. 5 shows one example of a thrombectomy device in which the distal capture region 506 is formed of a polyurethane membrane as mentioned above. In this example the distal capture region is a membrane, such as a porous membrane. The membrane may include small pores (e.g., between 100-200 microns and 5 mm, between about 0.1 mm and 5 mm, between about 0.2 mm and 5 mm, between about 0.1 mm and 3 mm, between about 1 mm and 5 mm, between about 1 mm and 3 mm, etc.). Thus, in some examples, the device may use a porous membrane formed of a polymeric material such as a porous polyurethane material, that is adhered to the distal end of the clot capture region (e.g., a stent) 502; the portion not covered by the membrane may be referred to as the clot engagement region 504.

As shown, the apparatus may include a control rod 509 from which the clot capture region extends. Any of these devices may also include a tip region (e.g., a microwire tip) 515 extending distally from the distal capture region 506, as shown. This microwire tip region 515 may help maintain tracking and position during deployment. In this example, the wire (microwire) tip region extending distally is a 0.014 microwire. The tip wire (microwire) may extend, e.g., between 1 and 100 mm (e.g., between 1 mm and 80 mm, between 1 mm and 70 mm, between 1 mm and 50 mm, between 1 mm and 40 mm, between 1 mm and 30 mm, between 1 mm and 20 mm, etc.).

A thrombectomy apparatus such as that shown in FIG. 5 may have different proportions (e.g., FIG. 5 is not to scale). The clot engagement region 504 may be approximately 35 mm long and the basket-like distal capture region may be about 15 mm long. The distal capture region 506 may have a porosity of the membrane that is greater than 100 micros (e.g., greater than 120 microns, greater than 140 microns, greater than 150 microns, greater than 170 microns, etc., such as between about 100-200 microns, between about 125-250 microns, between about 100-250 microns, etc.).

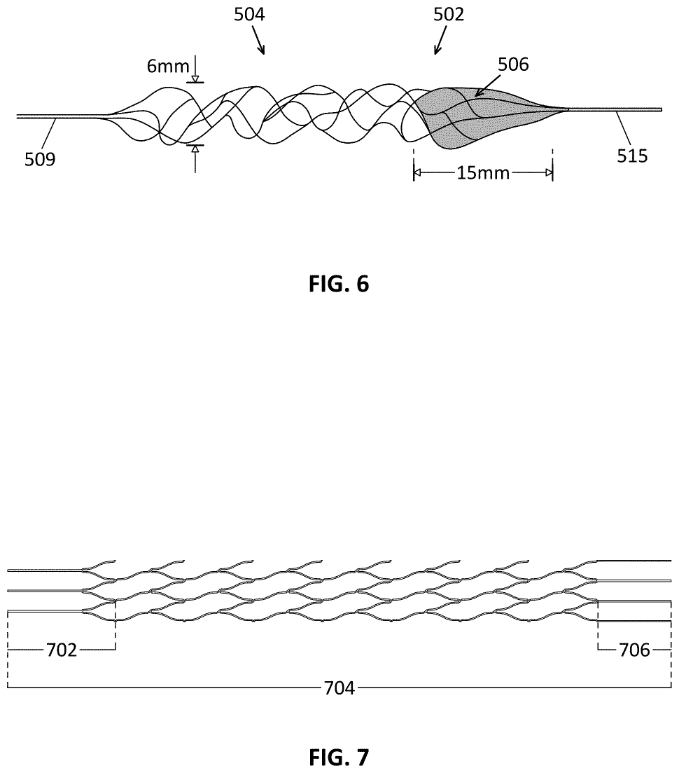

For example, FIG. 6 shows another example of a thrombectomy apparatus similar to that shown in FIG. 5 having dimensions shown; in an expanded version the clot engagement region 504 has a diameter of between about 4-12 mm (e.g., between about 5 mm-10 mm, between about 4 mm-8 mm, about 6 mm, about 7 mm, about 8 mm, about 5 mm or more, about 6 mm or more, about 7 mm or more, about 8 mm or more, etc.). The distal capture region 506 in this example has a length of approximately 15 mm along the long axis of the device. The polymeric (e.g., polyurethane) coating in this example is between 0.0005″ and 0.005″ (e.g., 0.001″) thick and may have a pore size of between about 0.050 mm to 5 mm, between about 0.1 mm to 5 mm, between about 0.1 mm to 3 mm, between about 1 mm to 5 mm, between about 1 mm to 3 mm, between about 0.3 mm to 3 mm, between about 0.1 mm-0.3 mm, between about 0.15 mm to 0.3 mm, between about 0.1 mm to 0.2 mm, between about 0.15 mm to 0.2 mm, between about 0.2 mm to 0.3 mm, etc.). The clot engagement region may be, e.g., between 10 mm and 70 mm long (e.g., between about 20-50 mm, between about 20-60 mm, between about 30-60 mm, etc., greater than 20 mm, greater than 30 mm, greater than 40 mm, greater than 50 mm, greater than 60 mm, etc.)

In general, the distal capture region in may include a hydrophilic coating to make it lubricious.

FIG. 7 illustrates one example of clot capture region formed of a stent, shown here in a flat configuration. The body of the clot capture region may be formed of a shape memory (e.g., superelastic) material such as nickel-titanium. The finished device may be electropolished. In this example, the overall length 704 is approximately 75 mm. This frame may be rolled up to form the stent-like body of the clot capture region and coated to form the distal clot capture region. In FIG. 7, the proximal end may form an attachment region 702 for attached to a rod or wire. The distal end, which will form the tapered distal capture region 706 may include a bonding region for forming the taper. This region, including the polymeric coating forming the membrane, may be formed on a mandrel, as described above.

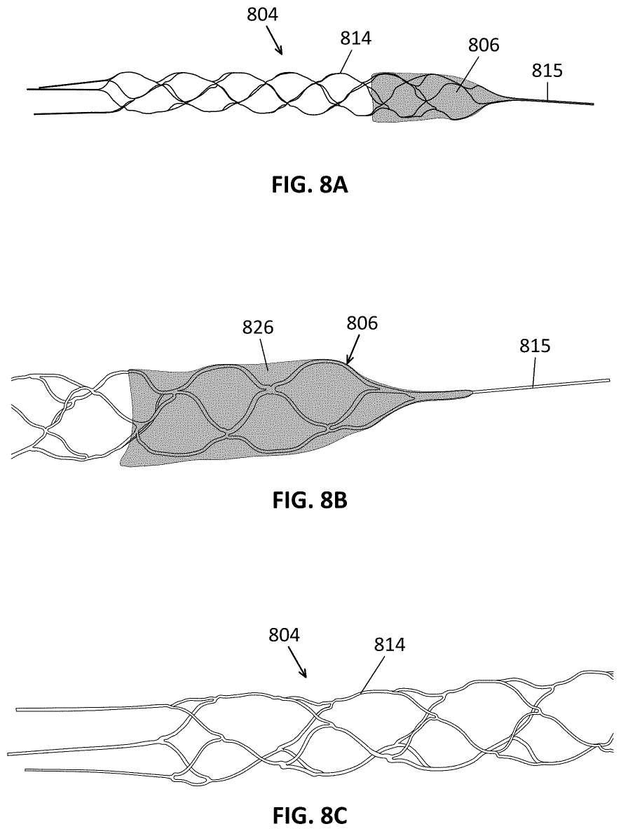

FIGS. 8A-8C illustrate another example of a thrombectomy apparatus. This example is similar to that shown in FIGS. 5 and 6, discussed above. In FIG. 8A, the apparatus includes a clot engagement region 804 that is formed from first region of a plurality of struts (814) and configured to expand with a first radial force. In this example, six struts (e.g., six wires, such as NITINOL or stainless steel wires) are shown; in some examples fewer or more than six (e.g., three, four, five, seven, eight, nine, ten, etc.) struts may be used. For example, eight or fewer struts may be used, seven or fewer struts, six or fewer struts, etc. The apparatus also includes a tapered distal capture region 806 at the distal end of the clot engagement region. The distal capture region may include a porous sheet of material, such as a polymeric membrane, that may be integrally formed onto the distal end region of the second region of struts. This distal end region may be referred to as a second region of a plurality of struts at a distal end of the clot engagement region. The second region of struts of the distal capture region may be configured sot that the struts are embedded within the porous material (e.g., within the polymeric membrane). The distal capture region may generally be configured to expand. The plurality of struts forming the distal capture region may be continuous with the plurality of struts forming the clot engagement region, or they may be separate. The plurality of struts forming the distal capture region may be the same number as the plurality of struts forming the clot engagement region (e.g., eight or fewer, seven or fewer, six or fewer, five or fewer, four or fewer, etc.), or they may be different. For example, the distal capture region may be formed of fewer struts than the clot engagement region. In some examples that distal capture region includes struts that have a different dimeter and/or cross-sectional shape as compared to the struts forming the clot engagement region.

In FIGS. 8A-8C, the distal clot capture region includes a sheet of porous material having pores configured as approximately 0.2 mm holes that are formed (e.g., by laser drilling) throughout the entire region. In some examples pores are only formed in the distal end of the distal clot capture region. In some examples pores of different sizes may be include (e.g., between 50 microns to 5 mm, between 0.1 mm to 5 mm, between 1 mm to 5 mm, between 1 mm to 3 mm, etc.). The density of pores may also be controlled, so that the pore density may be between 1% to 80% (e.g., between 5% to 75%, between 10% to 75%, between 15% to 70%, greater than 15%, greater than 30%, greater than 40%, greater than 50%, etc.). In some examples the pore density is between about 0.1 pore/mm² of membrane surface area and about 10 pore/mm² of membrane surface area (e.g., between about 0.5 pore/mm² and about 8 pore/mm², between about 0.5 pore/mm² and about 5 pore/mm², etc.).

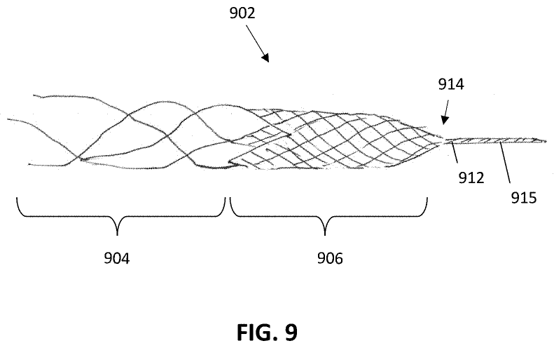

In some examples the apparatuses described herein may be formed by cutting the device out of a single piece of material (e.g., platinum, stainless steel, nitinol, etc.) such that a proximal portion of the device has larger struts and larger openings than the distal portion of the device, which is cut to have smaller openings. An example of this is shown in FIG. 9. FIG. 9 illustrates an example of an apparatus 902 (e.g., device) as described herein. In this example, the apparatus is formed from a sheet (or cylinder) of material, such as platinum, that has been laser cut to include a clot engagement region 904 having a plurality of struts that are configured to expand with a first radial force when compressed from the expanded configuration, as well as a tapered distal capture region 906 that includes pores that are formed into the clot engagement region. The pores in this example are also laser cut and may be, e.g., between about 0.1 mm and 5 mm (e.g., between about 1 mm and 3 mm, etc.). The distal capture region may also be formed into a plurality of struts. In some examples the distal end of the clot engagement region is also configured to expand radially with a second radial force when compressed from the expanded configuration. The device in FIG. 9 is shown in the expanded configuration. In FIG. 9, the distal capture region 906 narrows, shown in this example as a taper, to a smaller diameter at a distal end 914. The smaller distal end 914 may allow the distal capture region device to temporarily reside within smaller branches of at more distal portions of a blood vessel system and may otherwise help in steering the device. The example shown in FIG. 9 may also be coupled to a proximal control rod and/or wire (e.g., pull-wire), as described above. The device shown in FIG. 9 may also include one or more marker (e.g., a radiopaque marker) 912. The device shown in FIG. 9 also includes a distal microwire 915 extending from the tapered distal end region by between 0.5 cm and 10 cm. This microwire is pointed, beveled or otherwise configured to penetrate a clot.

In general, the struts forming the clot engagement region may be shaped to have a cross-sectional shape that is configured to engage with clot material. For example, the cross-sectional shape of the struts in at least the clot engagement region may be non-circular, including triangular, rectangular, etc. Thus the cross-sectional shape may be configured to cut into the clot material and may include one or more edges.

Any of these apparatuses may include a distal wire (e.g., distal microwire) 815 extending from the tapered distal end region. The distal wire may be configured to extend between about 0.5 cm and about 10 cm (e.g., between about 0.5 cm and about 5 cm, between about 0.5 cm and about 4 cm, between about 0.5 cm and about 3 cm, etc.) at the distal end of apparatus, as shown in FIG. 8B. The distal wire provides stability to the apparatus both before and during deployment. In particular, the distal wire has been found to enhance tracking within the deployment catheter and within the vessel. Distal wires that are between about 1 cm and about 5 cm have been found to be particularly effective as compared to shorter (e.g., less than 0.5 cm, less than about 1 cm, etc.) or longer (greater than 10 cm, etc.).

In FIG. 8B the distal capture region 806 includes the membrane 826 that is combined with the second region of the plurality of struts. In this example, the struts of the second region of the plurality of struts are embedded within the membrane. In some examples, the membrane is attached atop the struts of the second region. The second region of struts shown has a density that is slightly less than the density of the first region of struts of the clot engagement region 804, as shown in FIG. 8C. The lower density may allow packing and collapse of the distal end, including the membrane, before deploying the device, which may allow it to be more readily deployed within the body (e.g., through a delivery cannula).

As shown in FIG. 8C, the clot capture region may be formed of a first region of the plurality of struts that is biased (e.g., shape set) to expand when deployed. In FIG. 8C, the proximal end of the struts is shown loose; these struts may be gathered together, and may be coupled to an elongate member (e.g., wire, rod, catheter, etc.), such as to a control rod (not shown in FIG. 8C). In FIGS. 8B and 8C, the second region of struts has a greater density of struts than the first plurality of struts. As mentioned above, in some examples the density of struts may be less in the distal capture region as compared to the proximal clot engagement region.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or examples of various examples. Combinations of the above examples, and other examples not specifically described herein, will be apparent to those of skill in the art upon reviewing the above description.

What is claimed is:

1. A thrombectomy apparatus comprising:
a clot engagement region comprising a first region of a plurality of struts and configured to expand with a first radial force;
a tapered distal capture region comprising a porous region at a distal end of the clot engagement region, wherein the plurality of struts are continuous with the porous region, further wherein the tapered distal capture region is configured to expand radially with a second radial force, and wherein pores of the porous region are smaller than gaps between the struts of the clot engagement region; and
a distal microwire extending from the tapered distal capture region by between 0.5 cm and 10 cm.

2. The apparatus of claim 1, wherein the second radial force is less than the first radial force.

3. The apparatus of claim 1, wherein the clot engagement region is tapered to have a greater radial diameter at the distal end of the clot engagement region.

4. The apparatus of claim 1, wherein the porous region comprises a porous polymeric membrane having a porosity that is greater than 0.1 mm.

5. The apparatus of claim 1, wherein the clot engagement region and the tapered distal capture region are connected at a junction region, wherein struts of the first region are fused with struts of the tapered distal capture region at the junction region.

6. The apparatus of claim 1, wherein the clot engagement region and the tapered distal capture region are connected at a junction region, wherein the clot engagement region and then tapered distal capture region overlap at the junction region.

7. The apparatus of claim 1, wherein the clot engagement region has a longer length than the tapered distal capture region.

8. The apparatus of claim 1, wherein a distal end of the tapered distal capture region has a curved shape.

9. The apparatus of claim 1, wherein a distal end of the tapered distal capture region includes a dimple.

10. The apparatus of claim 1, wherein further comprising a first plurality of radio opaque markers at a proximal end of the clot engagement region and a second plurality of radio opaque markers at a junction between the clot engagement region and the tapered distal capture region.

11. The apparatus of claim 1, further comprising an elongated support extending through the clot engagement region and the tapered distal capture region and coupled to a distal end of the tapered distal capture region.

12. The apparatus of claim 1, wherein the clot engagement region is between 5 and 40 mm long and is between 1.1 and 2 times a length of the tapered distal capture region.

13. The apparatus of claim 1, wherein a maximum radial diameter of the clot engagement region is between 4 mm and 8 mm in an unconstrained state.

14. The apparatus of claim 1, further comprising a deliver catheter or sheath.

15. The apparatus of claim 1, further comprising a lubricious coating at the distal capture region.

16. The apparatus of claim 1, wherein the tapered distal capture region has a greater density of struts than the first region of the plurality of struts.

17. A method of removing a clot from a blood vessel, the method comprising:
passing a thrombectomy apparatus in a collapsed and constrained state through the clot within the blood vessel, wherein the thrombectomy apparatus comprises: a clot engagement region comprising a plurality of struts wherein the clot, a tapered distal capture region comprising a porous region at a distal end of the clot engagement region, wherein the plurality of struts are continuous with the porous region, and wherein pores of the porous region are smaller than gaps between the struts of the clot engagement region, and a distal microwire extending from the tapered distal capture region by between 0.5 cm and 10 cm; and
expanding the clot engagement region within the blood vessel with a first radial expansion force and expanding the tapered distal capture region within the blood vessel with a second radial expansion force, so that the clot engagement region expands through the clot, wherein the clot engagement region expands with a greater radial expansion force than the tapered distal capture region.

18. The method of claim 17, further comprising pulling the apparatus proximally to remove the clot from the blood vessel.

19. The method of claim 18, wherein the tapered distal capture region retains material from the clot during expansion of the clot engagement region and/or during removal of the apparatus from the blood vessel.

20. The method of claim 17, wherein expanding the clot engagement region and tapered distal capture region within the blood vessel comprises pulling a delivery catheter or sheath proximally to allow the tapered distal capture region and clot engagement region to expand radially, wherein a greater expansion force of the clot engagement region causes the clot engagement region to be pinned against walls of the blood vessel.
